(19) United States
(12) Patent Application Publication
(10) Pub. No.: US 2008/0133001 A1
(43) Pub. Date: Jun. 5, 2008

(54) Plastically deformable compositions and uses thereof

(75) Inventors: Alon Shalev, RaAnana (IL); Alexander Dubson, Petach-Tikva (IL)

Correspondence Address: Martin D. Moynihan;PRTSI, Inc., P.O. Box 16446, Arlington, VA, 22215, US

(73) Assignee: Nicast Ltd., Lod (IL)

(21) Appl. No.: 11/902,890

(22) Filed: Sep. 26, 2007

Related U.S. Application Data
(60) Provisional application No. 60/872,500, filed on Dec. 4, 2006.

(51) Int. Cl. A61F 2/82; A01N 1/00

(52) U.S. Cl. 623/1.49; 523/112

(57) ABSTRACT

A composition-of-matter, comprising one or more plastically deformable fiber is disclosed. The plastically deformable fiber(s) comprise a first and a second composition, where the first composition comprises at least one generally nondistensible polymer and the second composition comprises at least one agent capable of modulating distensibility of the generally nondistensible polymer(s).

### RELATED APPLICATION

This Application claims the benefit of U.S. Provisional Patent Application No. 60/872,500 filed on Dec. 4, 2006, the contents of which are hereby incorporated by reference in their entirety.

### FIELD AND BACKGROUND OF THE INVENTION

The present invention relates to a composition-of-matter and a medical device incorporating the composition-of-matter. More particularly, but not exclusively, the present invention relates to a medical device that can be used as a liner to a blood vessel.

Atheromatous plaques are accumulations of inflammatory cells, lipids, and connective tissue in arterial walls between the endothelium lining and the smooth muscle wall. Atherosclerosis is the result of development of atheromatous plaque, and is the leading cause of death in Western societies, causing heart attacks, strokes, and other cardiovascular problems.

Plaques can grow to a point where they obstruct blood flow through the artery, a process termed stenosis. Treatment of atherosclerosis has been focused to a considerable degree on solving the problem of stenosis. Hence, bypass surgery is aimed at directing blood flow around stenoses, and angioplasty, using increasingly sophisticated stents, is aimed at opening stenoses. Drug-eluting stents are now used to gradually release drugs that prevent reoccurrence of stenosis, termed restenosis.

However, stenosis is not the primary mechanism by which plaques endanger health. The main danger of plaques is that they may rupture, releasing debris and initiating thrombosis. The resulting blood clot may block blood flow, or debris from the plaque or clot may block smaller blood vessels downstream, which may result in a heart attack or stroke. Thus, plaques are particularly dangerous when they are most vulnerable to rupture, such as rapidly growing plaques with a thin cover. Because the arterial wall is pushed outwards as a plaque grows, such vulnerable plaques usually do not narrow the artery considerably, and in some cases may even widen the artery by creating an aneurysm. Treatments against stenosis do not address the problem of plaque rupture. On the contrary, angioplasty typically increases the danger, because when the artery is widened the plaque is oftentimes ruptured and debris thereof begin to drift downstream in the blood vessel. Furthermore, the materials of which the stents are composed frequently promote thrombosis.

Atheromatous plaques lead in some cases to turbulent blood flow. This turbulence can encourage further plaque formation and cause deterioration of the endothelial covering of the plaque, increasing the likelihood of plaque rupture.

Atheromatous plaque growth can also lead to aneurysm of the artery. The pressure of blood flow on the aneurysm can cause a hemorrhage, which can lead to serious debility or rapid death. The use of vascular grafts to safely direct blood past an aneurysm has been proposed. These grafts are similar to stents, in that they are tubular structures that expand to fill the artery, except that the purpose is not to hold the artery open, but to separate between the blood flow and the aneurysm in the arterial wall. One drawback of such vascular grafts is that it is difficult to anchor them in place. Blood may leak between the graft and the arterial wall into the aneurysm, defeating the purpose of the graft.

Many types of vascular grafts that can expand to form a tubular structure filling a blood vessel are known in the art. In general, such devices contain an expandable frame such as a stent, typically composed of a metal coil or wire mesh, which provides structural support. The frame expands either by its own spring-force or shape memory, or by application of pressure, typically by a balloon catheter. An outer layer can be adhered the frame to surround it in such a way that expansion of the frame holds the outer layer against the vascular wall. Fixed-radius outer layers are known in the art, but these are difficult to use because the outer layer must be manufactured with the exact radius of the blood vessel to avoid an imperfect fit. Expandable outer layer are thus preferable, as they can be expanded in situ to the desired radius. It is generally accepted that a porous outer layer is desirable in order to promote normal cell colonization on the outer face of the graft.

In addition, in many cases it is desirable to have a liner covering the anterior of the frame for separating the frame from the blood. This prevents adverse reactions, such as thrombosis, with the metallic frame, and prevents leakage of blood through the porous outer layer. Once the frame is expanded, e.g., via balloon catheter, the outer layer and/or liner expand therewith and remain in the expanded state due to the adherence forces between the liner and the frame and due to pressure applied by the expanded frame on the outer layer. Expandable outer layers and liners must be therefore capable of undergoing considerable expansion without tearing.

As examples of devices described above, U.S. Pat. Nos. 6,165,212 and 6,139,573 teach expandable supporting frames coated by an outer coat and lined by an inner liner.

U.S. Pat. No. 6,689,162 teaches a device containing structural strands interbraided with textile strands in order to combine the structural support of stents with the impermeability of a liner.

A device combining both structural supporting components and a coating material is complex, making it difficult to manufacture, and increasing the likelihood of failure. In addition, such devices are fairly cylindrical, and do not match the exact dimensions of the blood vessel. This makes it difficult to anchor such devices in place, and to prevent leakage of blood between the outer layer and the vascular wall, which would defeat the purpose of the liner.

A promising manufacturing technique for vascular grafts and other implantable devices is electrospinning. Electrospinning is a method for the manufacture of ultra-thin synthetic fibers which reduces the number of technological operations and increases the stability of properties of the product being manufactured. In regard to vascular prostheses, electrospinning and electrospinning-like manufacturing methods are disclosed, for example, in U.S. Pat. Nos. 4,562,707, 4,645,414, 5,639,278, 5,723,004 and 5,948,018. According to the electrospinning method, fibers of a given length are formed during the process of polymer solution flow from capillary apertures under electric forces and fall on a receptor to form a non-woven polymer material, the basic properties of which may be effectively altered.

The electrospinning technique has been employed for manufacturing various medical implants.

Devices using curable liquids have also been disclosed. For instance, U.S. Pat. No. 7,060,087 teaches a graft made of spun fibers or filaments and encapsulated biocompatible adhesive which glues the graft to the vascular wall. WO 2006/087721 teaches a graft device that uses a curable liquid to make the graft device rigid. However, liquids are inherently more difficult than solids to control, and may lead to adverse effects when used in situ, for instance, by curing at the wrong time or by diffusing out of the graft.

### SUMMARY OF THE INVENTION

According to one aspect of the present invention there is provided a composition-of-matter comprises at least one plastically deformable fiber, the plastically deformable fiber comprises a first and a second composition, the first composition comprises at least one generally nondistensible polymer and the second composition comprises at least one agent capable of modulating distensibility of the generally nondistensible polymer(s).

According to further features in embodiments of the invention described below, the generally nondistensible polymer is capable of withstanding tension of at least 8 MPa at a tensile strain of less than 22%.

According to still further features in the described embodiments agent(s) is/are selected such that the plastically deformable fiber(s) is/are capable of maintaining plastic deformation characterized by a strain of at least 300%.

According to still further features in the described embodiments the agent(s) comprises at least one elastic polymer.

According to still further features in the described embodiments the elastic polymer(s) is/are selected from the group consisting of a poly(ethylene-vinyl acetate), resilin, elastin, polyisoprene, a butyl rubber, a halogenated butyl rubber, polybutadiene, a styrene-butadiene copolymer, an acrylonitrile-butadiene copolymer, a hydrogenated acrylonitrile-butadiene copolymer, polychloroprene, an ethylene propylene copolymer, an ethylene propylene diene copolymer, an atactic polypropylene, a low-density polyethylene, a polymer or copolymer of epichlorohydrin, a polyacrylic rubber, a silicone rubber, a fluorosilicone rubber, a fluoroelastomer, a perfluoroelastomer, a chlorosulfonated polyethylene, a chlorinated polyethylene, a polyurethane rubber, a polysulfide rubber, a polyphosphazene, polynorbornene, an ethylene-acrylate copolymer, and stereoisomers, blends and copolymers thereof.

According to still further features in the described embodiments the elastic polymer has an elasticity of at least 50%.

According to still further features in the described embodiments the generally nondistensible polymer is selected from the group consisting of poly(butyl methacrylate), (PBMA), poly(methyl methacrylate) (PMMA), polyhydroxybutyrate (PHB) and polycaprolactone (PCL).

According to still further features in the described embodiments the composition-of-matter further comprises at least one fixation agent.

According to still further features in the described embodiments the fixation agent(s) is/are selected such that an overall elasticity of the composition-of-matter is less than 5%.

According to still further features in the described embodiments the fixation agent(s) comprises a polymer selected from the group consisting of poly(ethylene carbonate) (PEC) and poly(propylene carbonate) (PPC).

According to still further features in the described embodiments the first composition comprises at least one generally nondistensible polymer selected from the group consisting of poly(butyl methacrylate) (PBMA), polycaprolactone (PCL) and polyhydroxybutyrate (PHB), and the second composition comprises at least one elastic polymer selected from the group consisting of poly(ethylene-vinyl acetate) (EVA) and polybutadiene (PBD).

According to still further features in the described embodiments the composition-of-matter comprises PCL and EVA. According to still further features in the described embodiments the composition-of-matter further comprises PEC.

According to still further features in the described embodiments the composition-of-matter further comprises at least one plasticizing agent.

According to still further features in the described embodiments the plasticizing agent(s) is/are selected from the group consisting of paraffin oil, castor oil, propylene glycol, glycerin, sorbitol, erythritol, polyethylene glycol, an alkyl citrate, an alkyl sebacate, an alkyl azelate, an alkyl adipate, an acetylated monoglyceride and a surfactant.

According to still further features in the described embodiments about 50 weight percents of the plastically deformable fiber(s) biodegrade within a time period that ranges from 1 hour to 2 years.

According to still further features in the described embodiments the composition-of-matter is a single plastically deformable fiber.

According to still further features in the described embodiments the composition-of-matter comprises a plurality of plastically deformable fibers.

According to still further features in the described embodiments the plastically deformable fibers comprise non-woven fibers.

According to still further features in the described embodiments the non-woven fibers comprise electrospun non-woven fibers.

According to still further features in the described embodiments the composition-of-matter further comprises a third composition being attached to at least a part of a surface of the composition-of-matter.

According to still further features in the described embodiments the third composition comprises an agent for improving or inducing an adhesion of the composition-of-matter to a blood vessel.

According to still further features in the described embodiments the third composition comprises at least one gel-forming agent.

According to still further features in the described embodiments the gel-forming agent(s) is/are selected from the group consisting of a polypeptide, a polysaccharide, a hydrophilic polyacrylamide, a hydrophilic polyurethane, a hydrophilic polyacrylate, a hydrophilic polymethacrylate, and a hydrophilic silicone.

According to still further features in the described embodiments the composition-of-matter further comprises at least one pharmaceutically active agent incorporated therein.

According to still further features in the described embodiments the pharmaceutically active agent is selected from the group consisting of a therapeutically active agent and a diagnostic agent.

According to another aspect of the present invention there is provided a medical device, comprises a tubular structure adapted for being implanted in the vasculature of a mammal, the tubular structure being composed, at least in part, of the composition-of-matter described herein.

According to still further features in the described embodiments the plastic deformation comprises radial expansion of the tubular structure from a first diameter to a second diameter being larger than the first diameter.

According to still further features in the described embodiments the tubular structure is designed and constructed such that the radial expansion occurs under a pressure of less than 20 atmospheres.

According to still further features in the described embodiments the tubular structure is designed and constructed such that when the tubular structure is at the second diameter, the tubular structure is capable of maintaining a radial outward bias at a radial strain of less than 20% in response to an inward radial force of at least 0.1 Newtons per cm.

According to still further features in the described embodiments the device further comprises a balloon, wherein the tubular structure is mounted on the balloon.

According to still further features in the described embodiments the plastically deformable fiber(s) is biodegradable.

According to still further features in the described embodiments the composition-of-matter comprises a third composition being attached to at least a part of a surface thereof.

According to still further features in the described embodiments the tubular structure comprises at least one layer of plastically deformable fibers oriented predominantly circumferentially.

According to still further features in the described embodiments the tubular structure comprises at least one layer of plastically deformable fibers oriented predominantly longitudinally.

According to yet another aspect of the present invention there is provided a method of lining a blood vessel, the method comprises introducing the medical device described herein into the blood vessel.

According to still further features in the described embodiments the method further comprises inflating the balloon such as to expand the tubular structure.

According to still further features in the described embodiments the method further comprises imaging at least a part of the blood vessel during the introducing the medical device to the blood vessel.

According to still another aspect of the present invention there is provided a process of producing a composition-of-matter. The process comprises: mixing the generally nondistensible polymer(s) and the agent(s) so as to provide a liquefied mixture; and electrospinning the liquefied mixture onto a precipitation electrode such as to form at least one plastically deformable fiber, thereby forming the composition-of-matter.

According to still further features in the described embodiments the precipitation electrode comprises a rotating mandrel, thereby forming a tubular structure.

According to still further features in the described embodiments the process further comprises applying a thermal treatment to the tubular structure.

According to still further features in the described embodiments the thermal treatment is selected so as to enhance radial strength of the tubular structure.

According to still further features in the described embodiments the thermal treatment is selected so as to enhance anti-kinking resistance of the tubular structure.

According to still further features in the described embodiments the thermal treatment is selected so as to reduce a characteristic porosity of the tubular structure by at least 50%.

According to still further features in the described embodiments the thermal treatment is characterized by a temperature of from about 50° C. to about 60° C.

According to still further features in the described embodiments the thermal treatment comprises placing the tubular structure on a thermally isolated substrate and heating the tubular structure.

According to still further features in the described embodiments the thermal treatment comprises rolling the tubular structure on a heated plate.

According to still further features in the described embodiments the process further comprises mounting the tubular structure on a carrier device prior to the thermal treatment.

According to still further features in the described embodiments the process further comprises crimping the tubular structure on the carrier device.

According to still further features in the described embodiments the carrier device is the mandrel.

According to still further features in the described embodiments the process further comprises supplementing the liquefied polymer with a charge control agent, prior to the electrospinning.

The present embodiments successfully address the shortcomings of the presently known configurations by providing a composition-of-matter, medical device incorporating the composition-of-matter, method for producing the composition-of-matter and method for using the medical device.

Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, suitable methods and materials are described below. In case of conflict, the patent specification, including definitions, will control. In addition, the materials, methods, and examples are illustrative only and not intended to be limiting.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable subcombination.

As used herein, the singular form “a”, “an” and “the” include plural references unless the context clearly dictates otherwise. For example, the term “a protein” or “at least one protein” may include a plurality of proteins, including mixtures thereof.

As used herein the term “about” refers to ±10%.

Throughout this disclosure, various aspects of this invention can be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

Whenever a numerical range is indicated herein, it is meant to include any cited numeral (fractional or integral) within the indicated range. The phrases “ranging/ranges between” a first indicate number and a second indicate number and “ranging/ranges from” a first indicate number “to” a second indicate number are used herein interchangeably and are meant to include the first and second indicated numbers and all the fractional and integral numerals therebetween.

As used herein throughout, the term “comprising” means that other steps and ingredients that do not affect the final result can be added. This term encompasses the terms “consisting of” and “consisting essentially of”.

The phrase “consisting essentially of” means that the composition or method may include additional ingredients and/or steps, but only if the additional ingredients and/or steps do not materially alter the basic and novel characteristics of the claimed composition or method.

The term “method” or “process” refers to manners, means, techniques and procedures for accomplishing a given task including, but not limited to, those manners, means, techniques and procedures either known to, or readily developed from known manners, means, techniques and procedures by practitioners of the chemical, pharmacological, biological, biochemical and medical arts.

### BRIEF DESCRIPTION OF THE DRAWINGS

The invention is herein described, by way of example only, with reference to the accompanying drawings. With specific reference now to the drawings in detail, it is stressed that the particulars shown are by way of example and for purposes of illustrative discussion of the preferred embodiments of the present invention only, and are presented in the cause of providing what is believed to be the most useful and readily understood description of the principles and conceptual aspects of the invention. In this regard, no attempt is made to show structural details of the invention in more detail than is necessary for a fundamental understanding of the invention, the description taken with the drawings making apparent to those skilled in the art how the several forms of the invention may be embodied in practice.

In the drawings:

FIGS. 1a-d are schematic illustrations of a medical device, according to various exemplary embodiments of the present invention;

FIG. 2 is a schematic illustration of an exemplary procedure for thermally treating a tubular structure according to an embodiment of the present invention;

FIGS. 3a-b schematically illustrate a perspective view (FIG. 3a) and an enlarged section along line A-A (FIG. 3b) of a plate which can be used according to an embodiment of the invention for thermal treatment;

FIG. 3c is a schematic illustration of a tubular structure having sub-regions in which the local density is increased, according to various exemplary embodiments of the present invention;

FIGS. 4-5 are images of a section of tubular structures made of a plastically deformable composition, according to various exemplary embodiments of the present invention. The tubular structures are shown in their reduced state (right) and expanded state (left).

### DESCRIPTION OF THE PREFERRED EMBODIMENTS

The present embodiments comprise compositions-of-matter which can be used to manufacture various medical devices. Specifically, the present embodiments comprise compositions-of-matter made of one or more fibers which are designed capable of undergoing expansion in the form of plastic deformation, processes of preparing same, medical devices such as, for example, liners for blood vessels containing same and methods of lining a blood vessel utilizing same. The compositions-of-matter of the present embodiments allow to insert a narrow device into the vasculature of the patient and to expand the device to the desired width and shape at the proper location in the vasculature, while gradually and locally releasing beneficial drugs, inhibiting turbulent blood flow, preventing release of debris from plaques, and/or protecting aneurysms from rupture.

The principles and operation of a composition, device and method according to the present invention may be better understood with reference to the drawings and accompanying descriptions.

Before explaining at least one embodiment of the invention in detail, it is to be understood that the invention is not limited in its application to the details of construction and the arrangement of the components set forth in the following description or illustrated in the drawings. The invention is capable of other embodiments or of being practiced or carried out in various ways. Also, it is to be understood that the phraseology and terminology employed herein is for the purpose of description and should not be regarded as limiting.

As discussed hereinabove, the field of vascular grafts and of vascular aneurysms and atheromatous plaque, calls for the development of suitable materials and tubular structures made therefrom, which can satisfy the needs of modern medicine practices and research. These structures are often required to be made of biodegradable materials, which are non-toxic and benign both prior to the degradation process and thereafter (namely, have non-toxic and benign break-down products). These structures are further often required to contain and controllably release bioactive agents which are necessary for effecting the desired influence and activity of a particular device, prevent harmful effects which may be inflicted by the foreign implant and assist in the healing process. These structures should further preferably be characterized by mechanical and chemical properties that allow proper implantation and performance of the implanted structure. Specifically, it is desirable that the tubular structure is flexible enough to be capable of being shaped in situ to match the dimensions of the blood vessel, but be sufficiently resilient to preserve the final shape. In addition, it is desirable for the outer surface to be porous, for the inner surface to be smooth, and for the material to avoid adverse reactions with the body.

As discussed hereinabove, metal coils and wire meshes have been used as expandable structural supports for liner material having at least some of the desirable properties. However, the complexity of such combinations of structural supports and liner material make such devices sub-optimal.

In a search for a novel technique for constructing expandable tubular structures that could serve as vascular liners, the present inventors have devised and successfully created novel compositions that enable production of polymer fiber structures that are capable of undergoing expansion to the desired final shape without the aid of special structural support elements. The use of such fibers allows the adjustment of physical parameters such as porosity, strength, flexibility and distensibility, by altering the dimensions and density of the fibers of the material.

The compositions-of-matter obtained by this methodology are based on plastically deformable fibers which have sufficient distensibility for allowing the compositions to undergo expansion, but have sufficient plasticity to retain new configurations following expansion, thereby providing structural support for the expanded structure.

Formally, when a stress is applied to a material, such as a fiber, the stress tensor is a function of the strain tensor. A fiber can therefore be characterized in terms of a stress-strain curve which is a plot of a component of the stress as a function of the respective component of the strain.

The yield point of a material is commonly defined as the stress at which the individual molecular chains move in relation to one another such that when the pressure or stress is relieved, there is permanent deformation of the structure. When a material is subjected to pressure or stress below its yield point, the material typically follows the same stress-strain curve when subjected to multiple cycles of applying and relieving the stress or pressure. A material which exhibits the ability to follow the same stress-strain curve during the repeated application and relief of stress is typically referred to as being elastic and as having a high degree of elastic stress response.

Plasticity of a material is generally defined as the property which enables the material to be deformed without rupture during the application of a stress that exceeds its yield.

The term “elastic”, as used herein, describes a property of a material whereby the material may undergo deformation as a result of an applied stress, such that the material at least partially regains its original shape when the applied stress is removed. Thus, the deformation an elastic material exhibits is at least partially reversible.

The elasticity of a material is typically defined as the strain of the material at the yield point of the stress-strain curve.

The phrase “plastically deformable”, as used herein, describes a property of a material whereby the material may undergo deformation as a result of an applied force without breaking or tearing, such that the material retains a new shape after the applied force is removed.

The strain of a material is defined herein as the change in length of the material following application of stress divided by the original length of the material.

As is demonstrated and exemplified in the Examples section that follows, the present inventors have successfully produced tubular composite structures from polymeric fibers that can be expanded to obtain stable tubular composite structures with considerably larger radii.

Thus, according to various exemplary embodiments of the present invention there is provided a composition-of-matter that comprises one or more plastically deformable fiber(s). Each of the plastically deformable fibers composing the compositions-of-matter comprises at least one generally nondistensible polymer and at least one agent capable of modulating the distensibility of the generally nondistensible polymer(s).

The term “distensible”, as used herein, describes a property of a substantially compliant material whereby the material considerably stretches as a result of an applied tensile stress. It is appreciated that a distensible material can be either elastic or plastically deformable, depending whether the material regains its original or deformed shape when the applied tensile stress is removed.

The term “distensibility”, as used herein, refers to the maximal strain of the material during the application of a stress without being ruptured.

The phrase “generally nondistensible”, as used herein, describes a property of a material, wherein the material exhibits at most a relatively small amount of strain when subjected to a given amount of tensile stress. A generally nondistensible material is generally noncompliant to tensile stress but may or may not be compliant to shear stress.

In various exemplary embodiments of the invention the above mechanical characteristics (distensibility, non-distensibility, elasticity, plasticity, compliance, etc.) are associated with materials which are at room temperature or the body temperature of a mammal.

According to an embodiment of the present invention, the generally nondistensible polymer is capable of withstanding tension of at least 8 MPa at a tensile strain of less than 22% while being at a temperature of about 25° C.

The phrase “modulating distensibility”, as used herein, describes the increase of the distensibility of a first material by the addition of a second material, such that a composition comprising both materials is more distensible than the first material.

According to preferred embodiments of the present invention, the distensibility modulating agent is capable of increasing the distensibility of the fiber such that the fiber is capable of maintaining a distension characterized by a strain of at least 100%, more preferably at least 150%, more preferably at least 200%, more preferably at least 250% and optionally higher, e.g., at least 300%. In a preferred embodiment, the distension is a plastic deformation characterized by a strain of at least 300%.

The term “fiber”, as used herein, describes a class of structural elements, similar to pieces of thread, that are made of continuous filaments and/or discrete elongated pieces.

The term “polymer”, as used herein, encompasses organic and inorganic polymers and further encompasses one or more of a polymer, a copolymer or a mixture thereof (a blend).

According to preferred embodiments of the present invention, the distensibility modulating agent is an elastic polymer.

Elastic polymers are known in the art as “elastomers” or “rubbers”. The elastic polymer of the present embodiments preferably has significant distensibility in order to be capable of undergoing significant elastic deformation without breaking or tearing. Typically, distensibility is obtained by the absence of either large side chains or crystallinity.

Large side chains tend to create entanglement of different polymer molecules with each other, whereas small or nonexistent side chains allow the different polymer molecules to pass over each other relatively easily, thus allowing a change in configuration of the polymer when stress is applied.

Crystallinity comprises an arrangement of polymer molecules tightly bound in a relatively orderly arrangement. The tight binding of the polymer molecules in a crystalline arrangement prevents polymer molecules from passing over each other. Many elastic polymers include irregular positioning of side chains along at least part of the polymer molecule. Irregularly positioning of side chains can be obtained for instance, by random copolymerization of two monomers. Irregularly positioned side chains are less adept than regularly positioned side chains at inducing the regular molecular configurations characteristic of crystalline regions.

In addition to being distensible, elastic polymers retain their original shape following deformation. Typically, this is a result of localized connections between the polymer molecules, such as cross-links. For instance, many polymers are cross-linked by heating the polymer with sulfur, a process known as “vulcanization”. Vulcanization results in certain places along the polymer molecule being linked by sulfur atoms to neighboring polymer molecules. These cross-links prevent complete rearrangement of the polymer molecules, and thus cause the elastic polymer to return to its original configuration when an applied stress is removed. Cross-links also increase the mechanical strength of a polymer. However, when the polymeric molecules are cross-linked at only a small number of locations, they retain sufficient freedom of movement to render the polymer flexible.

In addition to cross-linking, polymeric molecules may be locally connected by microscopically-sized crystalline regions. If part of a polymeric molecule is bound to neighboring molecules in a crystalline arrangement, the rest of the polymeric molecule may retain sufficient freedom of movement relative to neighboring molecules to provide the distensibility necessary for an elastic polymer. In some cases, the polymeric molecule may be a copolymer of a polymer that is relatively compatible with crystallinity and a polymer that is relatively incompatible with crystallinity.

Preferred polymers that are suitable for use in the context of the present embodiments are biocompatible and/or biodegradable.

The term “biodegradable” as used herein, describes a material that can decompose under physiological conditions into breakdown products. Such physiological conditions include, for example, hydrolysis (decomposition via hydrolytic cleavage), enzymatic catalysis (enzymatic degradation), and mechanical interactions. Biodegradability is commonly desired for substances implanted in the body, as biodegradation of the substance limits the insult to the body by the substance.

The term “biodegradable” as used herein, also encompasses the term “bioresorbable”, which describes a substance that decomposes under physiological conditions to break down to products that undergo bioresorption into the host-organism, namely, become metabolites of the biochemical systems of the host-organism.

The term “biocompatible”, as used herein, describes a substance that can be in contact with biological material such as blood and tissue, without inducing adverse reactions, such as undesired inflammatory reactions or toxicity. Biocompatibility is naturally highly desirable for any embodiment of the present invention in the form of an implant.

Representative examples of elastic polymers that can therefore be used in the context of the present embodiments include, without limitation, a poly(ethylene-vinyl acetate), resilin, elastin, polyisoprene, a butyl rubber, a halogenated butyl rubber, polybutadiene, a styrene-butadiene copolymer, an acrylonitrile-butadiene copolymer, a hydrogenated acrylonitrile-butadiene copolymer, polychloroprene, an ethylene-propylene copolymer, an ethylene-propylene-diene copolymer, an atactic polypropylene, a low-density polyethylene, a polymer or copolymer of epichlorohydrin, a polyacrylic rubber, a silicone rubber, a fluorosilicone rubber, a fluoroelastomer, a perfluoroelastomer, a chlorosulfonated polyethylene, a chlorinated polyethylene, a polyurethane rubber, a polysulfide rubber, a polyphosphazene, polynorbornene, an ethylene-acrylate copolymer. The polymers listed hereinabove are intended to encompass derivatives, stereoisomers, copolymers and blends of the polymers.

The term “poly(ethylene-vinyl acetate)”, as used herein, encompasses copolymers of ethylene and vinyl acetate. Such copolymers are relatively inert and biocompatible, and thus often used in implantations and other medical applications.

The term “polyisoprene”, as used herein, encompasses both synthetic compositions of polyisoprene and natural compositions of polyisoprene. Polyisoprene from natural sources such as latex, often includes impurities such as proteins and sugars.

Polyisoprene is not normally considered biocompatible although it has been reported to be biocompatible when free of impurities such as cross-linking agents and proteins. Polymers of chloroprene, butadiene and acrylonitrile have also been found to be biocompatible when free of impurities.

The term “butyl rubber”, as used herein, encompasses copolymers of isobutylene with a small amount of an additional monomer, the additional monomer typically being isoprene. Butyl rubbers may be reacted with a halogen to produce halogenated butyl rubbers.

The term “atactic”, as used herein, describes a lack of correlation of the orientation of the methyl side groups in polypropylene with the orientation of neighboring methyl groups in the polymer molecule. The irregular orientation of methyl groups in atactic polypropylene results in reduced crystallinity, and thus increased elasticity, compared to polypropylene with a more regular orientation of methyl groups. Polypropylene is biocompatible and is often used in implants.

The phrase “low-density polyethylene”, as used herein, describes polyethylene with a density between 0.91 and 0.94 grams per cubic centimeter. Typically, this form of polyethylene has a more branched molecular structure than other forms of polyethylene. The branched structure prevents efficient packing of different molecules with each other, which both reduces the density and the crystallinity of the material. Polyethylene is biocompatible and is often used in implants.

The term “polyacrylic”, as used herein encompasses polymers of esters of acrylic acid, and is synonymous with the term “polyacrylate”. Some polyacrylic compounds, such as poly(methyl acrylate) and poly(ethyl acrylate), have been used in implants. The phrase “polyacrylic rubber”, as used herein, encompasses elastic polymers of esters of acrylic acid.

The term “fluoroelastomer”, as used herein, encompasses copolymers of vinylidene fluoride (VDF) and hexafluoropropylene (HFP); VDF, HFP and tetrafluoroethylene (TFE); VDF, HFP, TFE and perfluoromethylvinyl ether (PMVE); VDF, HFP, TFE, PMVE and ethylene; VDF, TFE and propylene; and TFE and propylene.

The term “perfluoroelastomer”, as used herein, encompasses copolymers of perfluorinated compounds such as TFE and PVE. Perfluoroelastomers, like other perfluorinated compounds, are inert, and therefore considered biocompatible.

The term “polyurethane”, as used herein, encompasses copolymers of a polyol and a diisocyanate, resulting in urethane links between the polyol and the diisocyanate. To obtain an elastic polymer, polyethylene glycol is typically used as the polyol. Polyurethanes have been used as biocompatible materials in medical devices.

The phrase “silicone rubber”, as used herein, encompasses elastic polymers with a backbone comprising alternating silicon and oxygen atoms, where two side groups are bound to the silicon atoms, the side groups being methyl groups, or methyl groups mixed with phenyl and/or vinyl groups. Silicone rubbers are inert and biocompatible, and thus frequently used in implants and other medical devices.

The phrase “fluorosilicone rubber”, as used herein, encompasses elastic polymers with a backbone comprising alternating silicon and oxygen atoms, where two side groups are bound to the silicon atoms, and where at least some of the side groups contain fluorine atoms. An example is silicone with methyl, vinyl and trifluoropropyl side groups. Fluorosilicones, like silicones, have been used in implants.

The phrase “chlorosulfonated polyethylene”, as used herein, encompasses polyethylenes to which sulfonyl chloride groups have been added, such as by reaction with chlorine and sulfur dioxide. Typically, chlorine groups are added along with the sulfonyl chloride groups to the polyethylene. Addition of chlorine groups alone results in chlorinated polyethylene. Because the added groups are randomly positioned, crystallinity is reduced.

The phrase “polysulfide rubber”, as used herein, encompasses elastic polymers comprising long chains of sulfur atoms terminated by carbon atoms. Typically, such polymers are synthesized by reacting sodium polysulfides with alkyl dihalides.

The term “polyphosphazene”, as used herein, encompasses polymers with a backbone comprising alternating phosphorus and nitrogen atoms, where side groups are attached to the phosphorus atoms. The properties of the polymer depend to a large extent on the properties of the side groups. Typically, a polyphosphazene is synthesized by first synthesizing a polyphosphazene in which the side groups are chlorine atoms, followed by substitution of the chlorine atoms with whatever side groups are desired. Random substitution of more than one type of side group can impart elasticity on the polymer by preventing crystallinity. Polyphosphazenes can also be made biodegradable by choosing a suitable side group.

According to preferred embodiments of the present invention, the elastic polymer has an elasticity of at least 50%, as defined herein, and thus can have an elasticity of, for example, 50%, 60%, 70%, 80%, 90%, 100% and even higher, for example, 200%, 300%, 500%, 800%, 1000% and even 1500%. In various exemplary embodiments of the invention the elastic polymer has an elasticity that ranges from about 400% to about 800%.

Many generally nondistensible polymers are contemplated. Representative examples include, without limitation, polypropylene, poly(vinyl chloride), poly(ethylene terephthalate), and polycarbonate make up almost 98% of the synthetic polymers encountered in daily life, and these are generally nondistensible polymers, although polyethylene, polypropylene and polycarbonate also have elastic forms (low-density polyethylene, atactic propylene and poly(ethylene carbonate)). Other examples of non-elastic polymers include, without limitation, polyamides, polyesters and polymethacrylates.

Examples of biodegradable, generally nondistensible polymers include, without limitation, poly(lactic acid), polycaprolactone, cellulose, poly(glycolic acid), polyhydroxybutyrate, polyhydroxyvalerate, polyhydroxyhexanoate, polyhydroxyoctanoate and zein.

Polyethylene, polypropylene, poly(ethylene terephthalate), polyesters such as poly(ether ether ketone) and biodegradable polyesters, polymethacrylates, some fluoropolymers and polycarbonates are non-limiting examples of polymers sufficiently biocompatible to have been used in implants. Polyamides have also been used for medical purposes requiring biocompatibility such as biocompatible nylon sutures.

Also contemplated are the following polymers: polystyrene, poly(vinyl chloride), cellulose, nitrocellulose and cellulose acetate.

The term “polycarbonate”, as used herein, encompasses polymers with a repeating —[R—O—C(═O)—O]— unit. Bisphenol A is commonly used as the R group.

The term “polyamide” is used herein and in the art to encompass polymers in which the monomers are bound to each other by an amide link, which is typically formed by the reaction of a carboxylic acid and an amine. Nylon, Kevlar and Nomex are commonly used polyamides.

The term “polyester” is used in the art to encompass polymers in which the monomers are bound to each other by an ester link. The ester may link two identical monomers, as in polycaprolactone, or two or more types of monomers, as in poly(ethylene terephthalate).

The term “polymethacrylate”, as used herein, encompasses polymers of esters of methacrylic acid.

According to the preferred embodiments of the present invention, the generally nondistensible polymer is selected from the group consisting of poly(butyl methacrylate) (PBMA), poly(methyl methacrylate) (PMMA), polyhydroxybutyrate (PHB) and polycaprolactone (PCL).

According to further preferred embodiments of the present invention, the composition-of-matter further comprises at least one fixation agent. A fixation agent may be any agent that increases the plasticity and decreases the elasticity of the deformation of the composition-of-matter when a given stress is applied.

Preferably, the fixation agent is capable of reducing the elasticity of the composition-of-matter to less than 10%. More preferably, the fixation agent is capable of reducing the elasticity of the composition-of-matter to less than 5%.

According to preferred embodiments of the present invention, the fixation agent(s) comprises at least one polymer. According to further preferred embodiments, the polymer is selected from the group consisting of poly(ethylene carbonate) (PEC) and poly(propylene carbonate) (PPC).

The terms “poly(ethylene carbonate)” and poly(propylene carbonate), as used herein, describe polymers comprising ethylene (CH₂CH₂) or isopropylene (CH₂CHCH₃) units respectively alternating with carbonate (CO₃) units, which may be produced by reacting carbon dioxide with ethylene (or propylene) oxide. These polymers have been found to be biodegradable and biocompatible.

According to preferred embodiments of the present invention, the composition-of matter comprises at least one generally nondistensible polymer selected from the group consisting of poly(butyl methacrylate) (PBMA), polycaprolactone (PCL) and polyhydroxybutyrate (PHB), and at least one elastic polymer selected from the group consisting of poly(ethylene-vinyl acetate) (EVA) and polybutadiene (PBD). Preferably, the composition-of matter comprises PCL and EVA. More preferably, the composition-of-matter comprises PCL, EVA and PEC.

The ratio of the generally nondistensible polymer to the distensibility modulating agent composing the fibers described herein can be determined by considering the original distensibility of the generally nondistensible polymer and the desired distensibility of the composition, namely, the capability of the composition to undergo deformation. This ratio is typically from about 1:1 to about 40:1 (by weight).

Preferably, the ratio of the generally nondistensible polymer to the distensibility modulating agent ranges from about 2:1 to about 30:1, more preferably from about 5:1 to about 20:1 or from about 5:1 to about 10:1.

Without being bound to any particular theory, it is assumed that molecules of the distensibility-modulating agent sufficiently separate between the molecules of the generally nondistensible polymer to allow distension under stress, while substantially retaining the mechanical strength typical of generally nondistensible polymers. Following creation of strain, molecules of the fixation agent, when present, become entwined with the molecules of the distensibility-modulating agent, thus fixating the molecules of the distensibility-modulating agent and nondistensible polymers to their deformed relative locations and rendering fiber plastically deformable.

The present inventors successfully prepared and characterized a composition-of-matter made of fibers containing polycaprolactone, poly(butyl methacrylate) and ethylene-vinyl acetate copolymer.

Additionally, the present inventors successfully prepared and characterized a composition-of-matter made of fibers containing polycaprolactone and poly(ethylene-vinyl acetate).

Additionally, the present inventors successfully prepared and characterized a composition-of-matter made of fibers containing polycaprolactone and poly(ethylene carbonate).

Additionally, the present inventors successfully prepared and characterized a composition-of-matter made of fibers containing polycaprolactone, ethylene-vinyl acetate copolymer and poly(ethylene carbonate).

In various exemplary embodiments of the invention the composition further comprises a plasticizing agent.

The term “plasticizing agent”, as used herein, describes compounds that soften polymeric materials when added to them. More specifically, a plasticizing agent enables stable stretching to a high stretching ratio during the application of a stress exceeding the yield of the composition.

While many plasticizing agents are considered as harmful agents when in the body, in applications that involve applying the compositions-of-matter described herein, either per se or within a medical device, the plasticizing agents are preferably selected such that cause minimal harm to the body and can be considered biocompatible.

Thus, exemplary plasticizing agents that are suitable for use in this context of the present invention include, without limitation, silicone oil, paraffin oil, castor oil, propylene glycol, glycerin, sorbitol, erythritol, polyethylene glycol, polypropylene glycol, organic esters, and surfactants.

The phrase “organic esters”, as used herein, encompasses esters of organic acids. Many organic esters are known in the art as plasticizers, diesters and triesters are used in particular, and organic esters are often biodegradable. Esters of citric acid, sebacic acid, azelaic acid, adipic acid, and fatty acids are promising as biocompatible plasticizers. Plasticizers with exemplary biodegradability and biocompatibility are alkyl citrates and acetylated monoglycerides. Alkyl citrates used as plasticizers include, without limitation, triethyl citrate, acetyl triethyl citrate, tributyl citrate, acetyl tributyl citrate, trioctyl citrate, acetyl trioctyl citrate, trihexyl citrate, acetyl trihexyl citrate, butyryl trihexyl citrate and trimethyl citrate. Acetylated monoglyceride, an ester of glycerol with acetic acid and a fatty acid, is an accepted food additive, and is used also as a biodegradable and biocompatible plasticizer.

According to preferred embodiments of the present invention, the plastically deformable fiber(s) of the composition are biodegradable, as defined herein. According to further preferred embodiments of the present invention, 50% by weight of the plastically deformable fiber(s) undergo biodegradation within a time period ranging from 1 hour to 2 years.

The composition-of-matter described herein can be a single fiber, as described herein or, optionally and preferably, comprises a plurality of the fibers described herein. When comprising a plurality of fibers, the fibers can be the same or different.

The use of a plurality of fibers is especially advantageous in the context of the present embodiments, as a composition-of matter comprising fibers is deformable due both to the distensibility of the fibers and to relative movement of the fibers.

Preferably, the plurality of plastically deformable fibers comprises non-woven fibers. Electrospun non-woven fibers are an especially preferred embodiment of the present invention.

Electrospinning is a process used to form very thin fibers. A liquefied polymer (e.g., melted polymer or dissolved polymer) is extruded, for example under the action of hydrostatic pressure, through one or more capillary apertures which are typically in the shape of needles. As soon as meniscus forms from the extruded liquefied polymer, a process of solvent evaporation or cooling starts which is accompanied by the creation of capsules with a semi-rigid envelope or crust. An electric field, occasionally accompanied a by unipolar corona discharge in the area of the capillary apertures, is generated by a potential difference between the capillary apertures and a precipitation electrode. Because the liquefied polymer possesses a certain degree of electrical conductivity, the above-described capsules become charged. Electric forces of repulsion within the capsules lead to a drastic increase in hydrostatic pressure. The semi-rigid envelopes are stretched, and a number of point micro-ruptures are formed on the surface of each envelope leading to spraying of ultra-thin jets of liquefied polymer from the capillary apertures.

Under the effect of a Coulomb force, the jets depart from the capillary apertures and travel towards the opposite polarity electrode. Moving with high velocity in the inter-electrode space, the jet cools or solvent therein evaporates, thus forming fibers which are collected on the surface of the precipitation electrode. When the precipitation electrode rotates, the charged fibers can form a tubular shape.

It is expected that during the life of this patent many relevant variations of electrospinning will be developed and the scope of the term “electrospinning” is intended to include all such new technologies a priori.

An additional composition may be attached to the surface or part of the surface of the aforementioned composition of the present embodiments. The additional composition may be added in order to improve or induce adhesion of the first composition to the blood vessel, to lubricate the first composition, or to prevent irritation of the surrounding tissue. Gels and agents that form gels when exposed to water have been used to cover or partially cover medical devices inserted into the body.

Hydrophilic polymers are particularly useful for forming aqueous gels. Thus, exemplary gel-forming agents that were found suitable for use in the context of the present invention due to their gel-forming properties and biocompatibility are polypeptides, natural polysaccharides, derivatized polysaccharides, and hydrophilic polyacrylamides, polyurethanes, polyacrylates, polymethacrylates, and silicones.

Non-limiting examples of polypeptides that can be used to form aqueous gels include fibrin, collagen and gelatin.

Non-limiting examples of natural polysaccharides that can be used to form aqueous gels include agarose, alginic acid and hyaluronic acid.

Non-limiting examples of derivatized polysaccharides that can be used to form aqueous gels include carboxymethyl cellulose and hydroxypropylmethyl cellulose.

A non-limiting example of a hydrophilic polymethacrylate that can be used to form aqueous gels is poly(hydroxyethyl methacrylate).

Preferred examples of gel-forming agents are HydroSlip, comprising a hydrophilic polyurethane, and Hydron, comprising poly(hydroxyethyl methacrylate).

As mentioned hereinabove, vascular implants are often designed so as to contain pharmaceutically active agents. Thus, the composition of the present embodiments may include one or more pharmaceutically active agents.

The term “pharmaceutically active agent”, as used herein, includes any agent whose activity is medically beneficial, either by directly and beneficially acting on the body of the patient, or by aiding the treatment of the patient in any way, such as diagnostic agents.

Therapeutically active agents, which directly and beneficially act on the body of the patient, may include, without limitation, drugs, analgesics, receptor agonists, receptor antagonists, prostaglandins, cytokines, hormones, ion-channel activators, ion-channel blockers, nitric oxide donors, vaso-active agents, cardiovascular agents, vasodilators, anesthetics, enzymes, amino acids, peptides, proteins, enzyme activators, enzyme inhibitors, vitamins, cofactors, coenzymes, metabolites, anti-metabolic agents, non-steroidal anti-inflammatory drugs (NSAIDs), carnitine, anti-psychotic agents, anti-thrombogenic agents, anticoagulants, growth factors, statins, toxins, oligonucleotides, nucleic acids, antisense nucleic acids, antimicrobial agents, antibiotics, anti-viral agents, cytotoxic agents, anti-proliferative agents, chemotherapeutic agents, anti-diabetic agents, antibodies, antigens, phospholipids, polysaccharides, chelators and/or antioxidants.

Non-limiting examples of cardiovascular agents that can be beneficially incorporated in the composition of the present invention include adenosine, alteplase, amiodarone, anagrelide, argatroban, atenolol, atorvastatin, benazepril, captopril, carvedilol, cerivastatin, clonidine, clopidrogel, diltiazem, enalapril, fluvastatin, fosinopril, gemfibrozil, hydrochlorothiazide, irbesartan, lisinopril, lovastatin, mibefradil, oprelvekin, pravastatin, prazosin, quinapril, ramipril, simvastatin, terazosin, valsartan and verapamil.

Non-limiting examples of vasodilators that can be beneficially incorporated in the composition of the present invention include adenosine, doxazosin, prazosin, phenoxybenzamine, phentolamine, tamsulosin, alfuzosin, terazosin, L-arginine, bradykinin, endothelium-derived hyperpolarizing factor, histamine, niacin, nitroglycerin, isosorbide mononitrate, isosorbide dinitrate, pentaerythritol tetranitrate, sodium nitroprusside, sidenafil, tadalafil, vardenafil, platelet activating factor and prostacyclin.

Non-limiting examples of vitamins that can be beneficially incorporated in the composition of the present invention include vitamin A, thiamin, vitamin B₆, vitamin B₁₂, vitamin C, vitamin D, vitamin E, vitamin K, riboflavin, niacin, folate, biotin and pantothenic acid.

Non-limiting examples of metabolites that can be beneficially incorporated in the composition of the present invention include glucose, urea, ammonia, tartarate, salicylate, succinate, citrate, nicotinate etc.

Non-limiting examples of non-steroidal anti-inflammatory drugs that can be beneficially incorporated in the composition of the present invention include aspirin, celecoxib, diclofenac, diflunisal, etodolac, fenoprofen, flurbiprofen, ibuprofen, indomethacin, ketoprofen, ketorolac, meclofenamate, mefenamic acid, nabumetone, naproxen, oxaprozin, oxyphenbutazone, phenylbutazone, piroxicam, rofecoxib sulindac and tolmetin.

Non-limiting examples of anti-thrombogenic agents that can be beneficially incorporated in the composition of the present invention include dipyridamole, tirofiban, aspirin, heparin, heparin derivatives, urokinase, rapamycin, PPACK (dextrophenylalanine proline arginine chloromethylketone), probucol, and verapamil.

Non-limiting examples of antimicrobial agents that can be beneficially incorporated in the composition of the present invention include iodine, chlorhexidene, bronopol and triclosan.

Non-limiting examples of chemotherapeutic agents that can be beneficially incorporated in the composition of the present invention include amino containing chemotherapeutic agents such as daunorubicin, doxorubicin, N-(5,5-diacetoxypentyl)doxorubicin, anthracycline, mitomycin C, mitomycin A, 9-amino camptothecin, aminopertin, antinomycin, N⁸-acetyl spermidine, 1-(2-chloroethyl)-1,2-dimethanesulfonyl hydrazine, bleomycin, tallysomucin, and derivatives thereof; hydroxy containing chemotherapeutic agents such as etoposide, camptothecin, irinotecaan, topotecan, 9-amino camptothecin, paclitaxel, docetaxel, esperamycin, 1,8-dihydroxy-bicyclo[7.3.1]trideca-4-ene-2,6-diyne-13-one, anguidine, morpholino-doxorubicin, vincristine and vinblastine, and derivatives thereof, sulfhydril containing chemotherapeutic agents and carboxyl containing chemotherapeutic agents.

Non-limiting examples of anti-diabetic agents that can be beneficially incorporated in the composition of the present invention include lipoic acid, acarbose, acetohexamide, chlorpropamide, glimepiride, glipizide, glyburide, meglitol, metformin, miglitol, nateglinide, pioglitazone, repaglinide, rosiglitazone, tolazamide, tolbutamide and troglitazone.

Non-limiting examples of diagnostic agents that can be beneficially incorporated in the composition of the present invention include X-ray contrast agents such as iohexyl, iodixanol, ioversol, diatrizoate, metrizoate, ioxaglate, iopamidol and iopramide; MRI contrast agents such as gadodiamide, gadopentate and other compounds containing gadolinium; compounds containing radioactive isotopes such as radioactive technetium, thallium, indium, iodine, fluorine, rubidium, gallium and xenon; ultrasound contrast agents such as microbubbles containing air, nitrogen, sulfur hexafluoride or perfluorinated compounds; and dyes such as fluorescein.

As discussed hereinabove, the composition of the present embodiments as described herein can be designed to be suitable for use as a structural element in a medical device.

Hence, according to a further aspect of the present invention there is provided a medical device which comprises the composite structure described herein.

The present inventors, in developing the plastically deformable fibers and compositions-of-matter containing same, have envisioned a novel medical device that utilizes the mechanical properties of the plastically deformable fibers described herein.

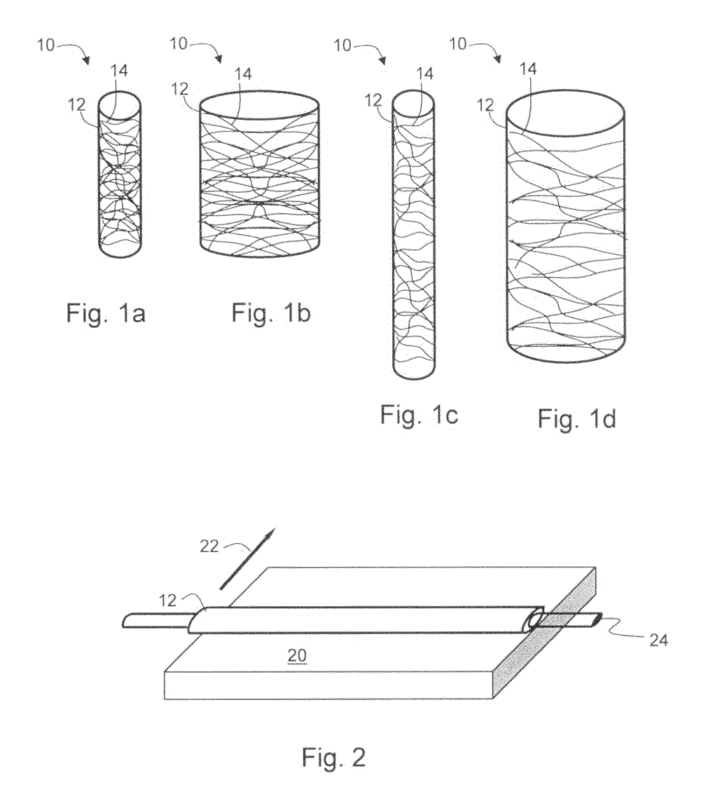

Reference is now made to FIGS. 1a-d which present schematic illustrations of a medical device 10, according to various exemplary embodiments of the present invention. The medical device is preferably in the form of a distensible tubular structure 12 made of a plurality of plastically deformable fibers 14. FIGS. 1a-d illustrate device 10 in its non-expanded state (FIG. 1a), an expand state be in which its diameter is increased (FIG. 1b), an expand state be in which its longitudinal dimension is increased (FIG. 1c), and an expand state be in which both its diameter and its longitudinal dimension are increased (FIG. 1d).

When subjected to sufficient increases in pressure within its interior, the tubular structure of the present embodiments expands so as to increase in an exterior dimension (diameter and/or length) by at least 250%, preferably 300%, and more preferably 400%, without rupturing. For example, it has been demonstrated that the exterior diameter of a tubular structure made of the plastically deformable fibers of the present embodiments can expand from about 1.35 mm to about 6 mm without rupturing in response to an internal pressure. It has further been demonstrated that the tubular structure substantially retains its expanded diameter even once the internal pressure is reduced to atmospheric pressure.

Such a tubular structure can be surgically inserted into the blood vessels of a patient when in a state where the tubular structure is narrower than the blood vessels. When the tubular structure is placed at a location where it is desirable to provide a lining for a blood vessel, the tubular structure can be widened, for example, until the dimensions match those of the inner wall of the blood vessel. When the tubular structure is widened until coming into contact with the inner wall of the blood vessel, the final conformation of the tubular structure can accurately match the conformation of the blood vessel. When the tubular structure is further widened to a diameter which is larger than the diameter of the blood vessel, the tubular structure widens the blood vessel. The plastically deformable nature of the composition of which the tubular structure is comprised allows the tubular structure to retain the desired shape after being expanded.

Thus, according to another aspect of the present invention there is provided a medical device comprising a tubular structure adapted for being implanted in the vasculature of a mammal. The tubular structure comprises the plastically deformable composition described herein.

It is desirable that the tubular structure undergo expansion when subjected to a stress that is weak enough to be applied easily inside the blood vessel. It is also desirable that the expanded tubular structure be strong enough to withstand inward radial forces such as those applied by the wall of the blood vessel. One skilled in the art would appreciate the trade-off between the two above factors, and would be capable of selecting the appropriate mechanical parameters for the tubular structure according to the intended function and circumstances of the use of the tubular structure.

Thus, according to preferred embodiments of the present invention, the tubular structure is designed to be capable of undergoing radial expansion under a pressure of less than 20 atmospheres, more preferably less than 19 atmospheres, more preferably less than 18 atmospheres, more preferably less than 17 atmospheres, more preferably less than 16 atmospheres, more preferably less than 15 atmospheres, e.g., about 14 atmospheres. In alternative embodiments, the tubular structure is capable of undergoing radial expansion under a pressure of less than 10 atmospheres.

According to further preferred embodiments of the present invention, the tubular structure, when expanded, is capable of maintaining a radial outward bias at a strain of less than X in response to an inward radial force of at least 0.1 Newtons per cm, where X is about 45%, more preferably about 40%, more preferably about 30%, more preferably about 20%, e.g., about 10% or less.

The device of the present embodiments can be introduced into the body in a narrow, compact state, and expanded when in place to fill the blood vessel. One method for expanding the device of the present embodiments is by inflation of a balloon inside the device.

Therefore, according to various exemplary embodiments of the present invention, the medical device comprises a balloon.

The mechanical properties of a structure comprising fibers depend in part on the orientation of the fibers. Circumferentially oriented fibers provide a tubular structure with resistance to radial compression, whereas longitudinally oriented fibers provide longitudinal strength. The relative degree of strength and flexibility required by an implanted tubular structure may vary according to different situations. The desired strength and flexibility may be obtained by orienting the fibers predominantly in a particular direction. Thus, the medical device of the present embodiments may be produced such that the plastically deformable fibers are oriented predominantly circumferentially, or such that the plastically deformable fibers are oriented predominantly longitudinally.

As discussed hereinabove, the medical device of the present embodiments is designed to be suitable for use according to a particular method of treatment.

Hence, according to a further aspect of the present invention there is provided a method of lining a blood vessel, the method comprising introduction of the medical device described herein into the blood vessel.

The term “lining”, as used herein, describes the process of covering a section of the inside wall of a blood vessel with a layer of material, without significantly impeding blood flow. As described hereinabove, reasons for lining a blood vessel include, but are not limited to, treating atheromatous plaque and vascular aneurysms, and facilitating localized drug release.

According to the preferred embodiments of the present invention, the method comprises inflating a balloon upon which the tubular structure of the medical device is mounted.

The method may further comprise imaging at least the respective portion blood vessel in order to facilitate proper placement and expansion of the medical device. Examples of imaging techniques include, without limitation, magnetic resonance imaging, X-ray imaging, ultrasound imaging, and gamma ray and positron emission techniques. Imaging may be performed with the aid of diagnostic agents, as discussed hereinabove. The most beneficial use of imaging in the context of the present invention is expected to be addition to the blood vessel of the patient of a diagnostic agent such as a contrast agent, in order to present an image of the blood vessel while introducing the medical device into the blood vessel.

In order to produce the compositions described herein, and particularly such structures which combine desired properties such as plastic deformation and biodegradability, the present inventors have developed a novel process.

Thus, according to another aspect of the present invention there is provided a process of preparing the compositions described herein. The process is effected by mixing the generally nondistensible polymer and the agent which modulates distensibility in a liquefied mixture, and electrospinning the mixture onto a precipitation electrode. The resulting jets of liquefied polymer evaporate, thus forming the one or more plastically deformable fibers that the composition described herein comprises on the precipitation electrode. A typical thickness of the fibers thus formed ranges between 50 nanometers and 50 micrometers.

According to the preferred embodiments of the present invention, the process further comprises precipitating the mixture onto an electrode comprising a rotating mandrel.

According to preferred embodiments of the present invention, the process comprises applying a thermal treatment to the tubular structure. Preferably the thermal treatment is at a temperature from about 50° C. to about 60° C. For example, in one embodiment, the thermal treatment is at a temperature of about 55° C., in another embodiment, the thermal treatment is at a temperature of about 58° C.

Thermal treatment of the tubular structure may be performed according to any method known in the art.

For example, in one embodiment, the tubular structure is placed on a thermally isolated substrate and heated, e.g., in an oven.

In another embodiment, the thermal treatment comprises rolling the tubular structure on a heated plate. In this embodiment, the tubular structure can be mounted on a carrier prior to the rolling procedure. Alternatively, the tubular structure can be rolled on the heated plate while being still mounted on the mandrel. The procedure is schematically illustrated in FIG. 2, showing tubular structure 12 mounted on a carrier 24 and a heated plate 20. The direction of rolling is shown by an arrow 22.

In various exemplary embodiments of the invention a more perfect fit to carrier 24 is induced by crimping tubular structure 12 thereon. In the embodiments in which a thermal treatment is employed, the crimping is preferably done prior to the thermal treatment. The crimping can be done using a crimping device as known in the art (to this end see, e.g., U.S. Pat. Nos. 5,626,604, 6,024,737, 6,092,273 and 6,510,722). The crimping device can have smooth jaws or alternatively one or more of the jaws can have a non-smooth profile

Following the creping process, the porosity of the structure is typically reduced. For example, it was found by the present Inventors that when a structure having a porosity of about 80% is subjected to crimping, its porosity is reduced to about 30%.

Tubular structure formed in a typical electrospinning process, may lack sufficient kinking resistance and further reinforcement of the final product is often necessary to support the lumen of the tubular structure while bending. According to an embodiment of the present invention the crimping and/or thermal treatment is applied so as to impart the tubular structure with intrinsic kinking resistance (e.g., without additional supporting elements). Thus, for example, the jaws of the crimping device can include a pattern selected such as to form grooved crimp on tubular structure 12, hence to provide a tubular structure having an alternating density in the longitudinal direction. More specifically, the jaws can have rings which form compressed sub-regions on the tubular structure, such that the basis weight of the sub-regions compressed by the rings is larger than the basis weight of sub-regions between adjacent rings of the crimping device.

Alternatively or additionally, the heated plate on which the thermal treatment is employed is formed such that when the tubular structure is rolled on the plate, the local density of the fibers is increased in a plurality of predetermined sub-regions along the structure resulting in a tubular structure having an alternating density in the longitudinal direction.

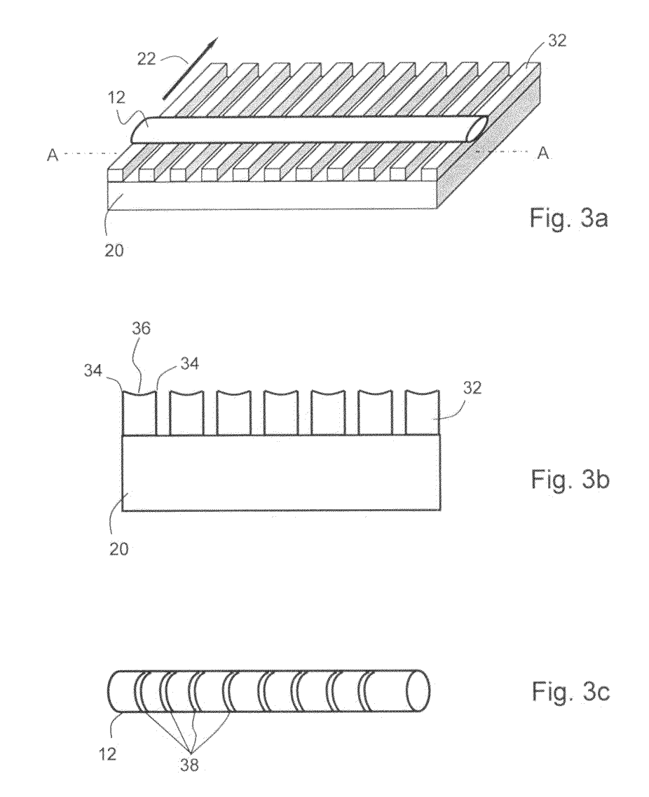

FIGS. 3a-b illustrate a perspective view (FIG. 3a) and an enlarged section along line A-A (FIG. 3b) of plate 20 according to the present embodiment of the invention. As shown, plate 20 is manufactured with an arrangement of ribs 32. Optionally the orientation of the ribs is parallel to the direction of rolling 22. The profile of the ribs can be a rectangular profile or it can have a different shape. In various exemplary embodiments of the invention the edges 34 of each rib are elevated relative to a central part 36 of the rib. An exemplary tubular structure manufactured in accordance with the present embodiment is illustrated in FIG. 3c, showing sub-regions 38 in which the local density is increased.

According to an embodiment of the present invention, the process further comprises supplementing the liquefied polymer mixture with a charge control agent.

The term “charge control agent”, as used herein described, describes an agent, such as a dipolar additive, that is added to the liquefied polymer in order to improve the behavior of the liquefied polymer under an electric field. It is assumed, in a non-limiting fashion, that the charge control agent improves the interaction between the polymer and ionized air molecules formed under the influence of the electric field, and that the extra charge thus attributed to the newly formed fibers is responsible for their more homogenous precipitation on the precipitation electrode. The charge control agent is typically added in the grams equivalent per liter range, say, in the range of from about 0.001 N to about 0.1 N, depending on the respective molecular weights of the polymer and the charge control agent used.

The method may further comprise providing a second electric field defined by a subsidiary electrode that is kept at a second potential difference relative to precipitation electrode. The purpose of the second electric field is to reduce non-uniformities in the first electric field so as to ensure a predetermined fiber orientation. The compositions described herein can thereby be made to comprise fibers oriented predominantly circumferentially and/or predominantly longitudinally. Additionally, the composite structure can be a multilayer structure in which one or more layers are characterized by a predetermined predominant fiber orientation.

The advantage of using a plurality of layers is that with such configuration each layer can have different properties, such as porosity, mechanical strength, and the like, depending on its function. For example, an inner layer can be manufactured substantially as a smooth surface with relatively low porosity. Such layer can prevent bleeding and preclotting and can ensure antithrombogenic properties and efficient endothelization. A typical thickness of such layer is from about 40 μm to about 80 μm. An outer layer of the structure can have thickness of from about 50 μm to about 1000 μm, so as to provide the structure with requisite mechanical properties.

Additional objects, advantages, and novel features of the present invention will become apparent to one ordinarily skilled in the art upon examination of the following examples, which are not intended to be limiting. Additionally, each of the various embodiments and aspects of the present invention as delineated hereinabove and as claimed in the claims section below finds experimental support in the following examples.

### EXAMPLES

Reference is now made to the following examples, which together with the above descriptions, illustrate the invention in a non limiting fashion.

### Material and Experimental Methods

Ethylene-vinyl acetate copolymer (EVA) with vinyl acetate content 33%, poly(butyl methacrylate) (PBMA), and polycaprolactone (PCL) were purchased from Scientific Polymer Products, Inc. Poly(ethylene carbonate) (PEC) was purchased from Empower Materials.

Mechanical characteristics were measured using the Hounsfield Test Equipment Ltd. H5KS model machine.

### Example 1

### Mechanical Tests for Various Compositions

Five types of composition-of-matters produced in accordance with various exemplary embodiments of the present invention were subjected to mechanical tests. Each composition-of-matters was in the form of a tubular structure composed of a plurality of plastically deformable fibers.

The liquefied polymer mixtures for the production of the tubular structures included PCL:EVA:PEC in a 9:0.5:0.5 weight ratio (sample No. 1), PCL:EVA:PEC in a 9:0.5:1 weight ratio (sample No. 2), PCL:EVA:PEC in a 8:1:1 weight ratio (sample No. 3), PCL:EVA:PEC in a 8.5:1:0.5 weight ratio (sample No. 4), and PCL:EVA:PEC in a 8.5:0.5:1 weight ratio (sample No. 5). The liquefied polymers were dissolved separately in chloroform at normal conditions during 24 hours by means of a magnetic stirrer and mixed. The solution was filtered and its conductivity adjusted up to about I μS. The solution concentration was 9%, and the viscosity was 560 cP.

All tubular structures were made via electrospinning. The electrospun tubular structures were subjected to thermal treatment by rolling them on a plate heated to 55° C.

The dimensions of all tubular structures were: 1.5 mm in inner diameter, 3.5 mm in outer diameter and 55 mm in length. The mechanical tests included stretching by applying opposite forces along the longitudinal axis of the tubular structures. The effective length of the tubular structures between the clamps of the test equipment was 10 mm.

The results of the mechanical tests for samples Nos. 1-5 are summarized in Table 1.

TABLE 1

| | maximal load at | maximal expansion at |
|---|---|---|
| sample No. | break [N] | break [mm] |
| 1 | 9.750 | 183.432 |
| 2 | 7.250 | 181.619 |
| 3 | 2.383 | 78.171 |
| 4 | 2.066 | 98.891 |
| 5 | 2.000 | 108.696 |

### Example 2

### PCL-EVA-PBMA, No Thermal Treatment

Ethylene-vinyl acetate copolymer (EVA), Poly(butyl methacrylate) (PBMA) and polycaprolactone (PCL), in a 0.5:0.5:9 weight ratio, were dissolved separately in chloroform at normal conditions during 24 hours by means of a magnetic stirrer and mixed. The solution was filtered and its conductivity adjusted up to about I μS. The solution concentration was 9%, and the viscosity was 560 cP.

The mixture was used as a liquid in an electrospinning process, in which polymer fibers were precipitated on a mandrel. The dimensions of the mandrel were about 2 mm in diameter and about 300 mm in length. The obtained tubular structure was characterized by porosity of about 80% and was further crimped by means of an MSI Stent Crimping Device. After crimping the product porosity was about 30%. No thermal treatment was applied.

The produced tubular structure was subjected to mechanical tests to determine the extension load at break, and the relaxation rate, defined as the change in strain following removal of load. The tubular structure was further subjected to an inward radial force so as to determine its capability to maintain a radial outward bias.

The extension at break was about 850%, the load at break was about 2.4 N, and the relaxation rate was about 11%. The tubular structure maintained a radial outward bias at a radial strain of about 32% in response to a localized inward radial force of about 0.1 Newtons/cm.

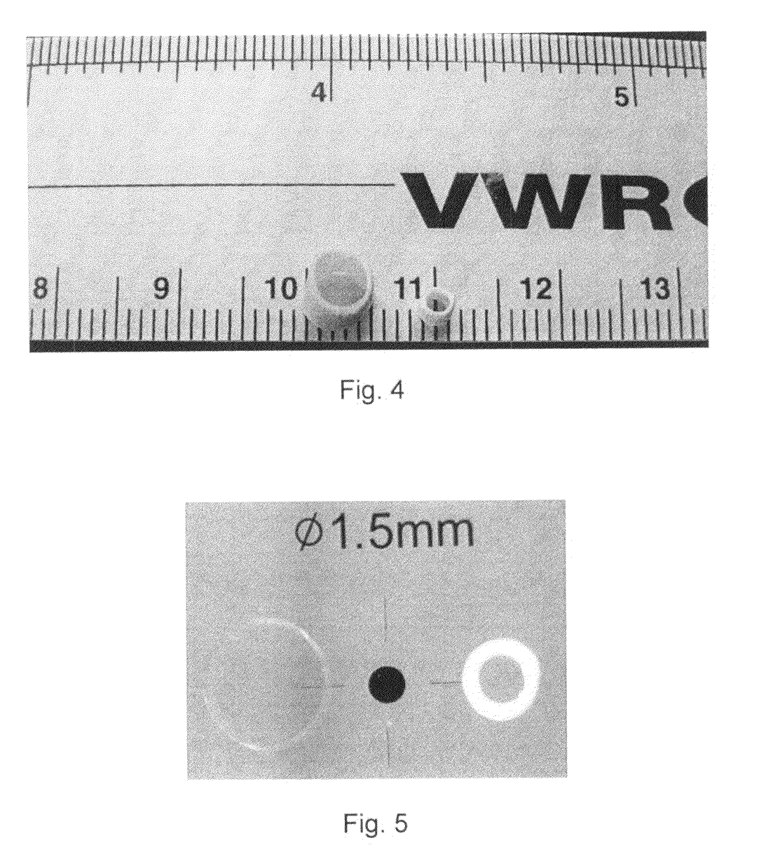

FIGS. 4-5 are images of a section of two tubular structures prepared according to embodiments of the present invention. Show are the tubular structures in the reduced state (right) and the expanded state (left). The mark in the middle of FIG. 5 is for comparison purpose. The diameter of the mark is 1.5 mm. As shown in FIG. 4, the diameter of the reduced state is about 2 mm and the diameter of the expanded state is about 5 mm, corresponding to a (linear) strain of about 250%. Even higher strain is demonstrated in FIG. 5. Each of the obtained structures were capable of maintaining the strain without metal support.

### Example 3

### PCL-EVA, No Thermal Treatment

Two tubular structures were manufactured from a mixture of polycaprolactone (PCL) and poly(ethylene-vinyl acetate) (EVA). For a first tubular structure the weight ratio of PCL:EVA was 9:1 and for a second tubular structure the PCL:EVA weight ratio was 9.5:0.5. The mixing and electrospinning were performed as described in Example 2 above.

Both tubular structures were characterized by porosity of about 80%. The tubular structures were further crimped as described in Example 2. After crimping the porosity was about 30% for both structures. For the first structure (9:1 weight ratio) the extension at break was about 950%, the load at break was about 2.1 N, and the relaxation rate was about 17%. For the second structure (9.5:0.5 weight ratio) the extension at break was about 680%, the load at break was about 2.7 N, and the relaxation rate was about 14%. In response to a localized inward radial force of about 0.1 Newtons/cm, the tubular structures maintained a radial outward bias at a radial strain of about 40% (first structure), and about 37% (second structure).

### Example 4

### PCL-EVA-PEC, No Thermal Treatment

A 8.5:0.5:1 weight ratio and a 8.5:0.5:0.5 weight ratio tubular structures were manufactured from a mixture of Polycaprolactone (PCL), poly(ethylene-vinyl acetate) (EVA), and poly(ethylene carbonate) (PEC). The mixing and electrospinning were performed as described in Example 2 above, with no thermal treatment.

The tubular structures were characterized by porosity of about 80%. The tubular structures were further crimped as described in Example 2. After crimping, the porosity was about 30%. For the first structure (8.5:0.5:1 weight ratio) the extension at break was about 1000%, the load at break was about 3.5 N, and the relaxation rate was about 7%. For the second structure (8.5:0.5:0.5 weight ratio) the extension at break was about 1000%, the load at break was about 4 N, and the relaxation rate was about 4%. In response to an inward radial force of about 0.1 Newtons/cm, the tubular structures maintained a radial outward bias at a radial strain of about 42% (first structure) and about 33% (second structure).

### Example 5

### PCL-EVA-PEC, with Thermal Treatment

Six tubular structures were manufactured from a mixture of PCL, EVA and PEC at a weight ratio of 9:0.5:0.5, as described in Example 2 above. All six tubular structures were electrospun on a 1.5 mm mandrel which defined their inner diameter. Following electrospinning, the tubular structures were pressed on the mandrel by a crimping device. Two type of crimping device were employed: a smooth jaws device and a non-smooth jaws device. The jaws of the latter crimping device have 0.3×0.6 mm rings with a 1 mm step.

The tubular structures were subjected to four mechanical tests: (i) longitudinal stretching, (ii) radial expansion by a balloon, (iii) relaxation rate, and (iv) response to an inward radial force of 0.1 N/cm (stiffness).

Following is a description of the manufacturing process for each of the six tubular structures. The mechanical characteristics are summarized in Table 2, hereinunder.

### Tubular Structure No. 1

The electrospinning process was performed so as to provide an outer diameter of 3.4 mm and a linear density of 2.2 g/m. The tubular structure was further pressed by the smooth jaws crimping device so as to provide outer diameter of 2.5 mm. The crimped structure was subjected to thermal treatment by placing the structure in an oven heated to 55° C.

### Tubular Structure No. 2

The electrospinning process was performed so as to provide an outer diameter of 3.4 mm and a linear density of 2.2 g/m. The tubular structure was further pressed by the smooth jaws crimping device so as to provide outer diameter of 2.5 mm. The crimped structure was subjected to thermal treatment by placing the structure in an oven heated to 58° C.

### Tubular Structure No. 3

The electrospinning process was performed so as to provide an outer diameter of 4 mm and a linear density of 2.6 g/m. The tubular structure was further pressed by the smooth jaws crimping device so as to provide outer diameter of 2.5 mm. The crimped structure was subjected to thermal treatment by placing the structure in an oven heated to 55° C.

### Tubular Structure No. 4

The electrospinning process was performed so as to provide an outer diameter of 3.4 mm and a linear density of 2.6 g/m. The tubular structure was further pressed by the smooth jaws crimping device so as to provide outer diameter of 2.5 mm. The crimped structure was subjected to thermal treatment by placing the structure in an oven heated to 58° C.

### Tubular Structure No. 5

The electrospinning process was performed so as to provide an outer diameter of 3.4 mm and a linear density of 2.2 g/m. The tubular structure was further pressed by the non-smooth crimping device so as to provide outer diameter of 2.5 mm. The crimped structure was subjected to thermal treatment by placing the structure in an oven heated to 55° C.

### Tubular Structure No. 6

The electrospinning process was performed so as to provide an outer diameter of 3.4 mm and a linear density of 2.2 g/m. The tubular structure was further pressed by the non-smooth crimping device so as to provide outer diameter of 2.5 mm. The crimped structure was subjected to thermal treatment by placing the structure in an oven heated to 58° C.

Table 2 below summarizes the mechanical characteristics of the six structures.

TABLE 2

| | | longitudinal | balloon pressure | | |
|---|---|---|---|---|---|
| | Tension at | expansion | for radial | relaxation | |
| No. | break | at break | expansion | rate | stiffness |
| 1 | 4.2 N | 1100% | 16 bar | 8% | 25% |
| 2 | 9.2 N | 1460% | 20 bar | 5% | 20% |
| 3 | 5.8 N | 1200% | 18 bar | 8% | 22% |
| 4 | 11 N | 1600% | 23 bar | 5% | 6% |
| 5 | 4.5 N | 1100% | 18 bar | 6% | 8% |
| 6 | 12 N | 1240% | 18 bar | 8% | 10% |

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable subcombination.

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims. All publications, patents and patent applications mentioned in this specification are herein incorporated in their entirety by reference into the specification, to the same extent as if each individual publication, patent or patent application was specifically and individually indicated to be incorporated herein by reference. In addition, citation or identification of any reference in this application shall not be construed as an admission that such reference is available as prior art to the present invention.

What is claimed is:

1. A composition-of-matter comprising at least one plastically deformable fiber, said plastically deformable fiber comprises a first and a second composition, said first composition comprises at least one generally nondistensible polymer and said second composition comprises at least one agent capable of modulating distensibility of said at least one generally nondistensible polymer.

2. The composition-of-matter of claim 1, wherein said generally nondistensible polymer is capable of withstanding tension of at least 8 MPa at a tensile strain of less than 22%.

3. The composition-of-matter of claim 1, wherein said at least one agent is selected such that said at least one plastically deformable fiber is capable of maintaining plastic deformation characterized by a strain of at least 300%.

4. The composition-of-matter of claim 1, wherein said at least one agent comprises at least one elastic polymer.

5. The composition-of-matter of claim 4, wherein said elastic polymer has an elasticity of at least 50%.

6. The composition-of-matter of claim 1, wherein said generally nondistensible polymer is selected from the group consisting of poly(butyl methacrylate), (PBMA), poly(methyl methacrylate) (PMMA), polyhydroxybutyrate (PHB) and polycaprolactone (PCL).

7. The composition-of-matter of claim 1, further comprising at least one fixation agent.

8. The composition-of-matter of claim 7, wherein said at least one fixation agent is selected such that an overall elasticity of the composition-of-matter is less than 5%.

9. The composition-of-matter of claim 1, wherein said first composition comprises at least one generally nondistensible polymer selected from the group consisting of poly(butyl methacrylate) (PBMA), polycaprolactone (PCL) and polyhydroxybutyrate (PHB), and said second composition comprises at least one elastic polymer selected from the group consisting of poly(ethylene-vinyl acetate) (EVA) and polybutadiene (PBD).

10. The composition-of-matter of claim 9, further comprising PEC.

11. A medical device, comprising a tubular structure adapted for being implanted in the vasculature of a mammal, said tubular structure being composed, at least in part, of the composition-of-matter of claim 1.

12. The device of claim 11, wherein said plastic deformation comprises radial expansion, from a first diameter to a second diameter being larger than said first diameter.

13. The device of claim 11, wherein said tubular structure is designed and constructed such that said radial expansion occurs under a pressure of less than 20 atmospheres.

14. The device of claim 12, wherein said tubular structure is designed and constructed such that when said tubular structure is at said second diameter, said tubular structure is capable of maintaining a radial outward bias at a radial strain of less than 20% in response to an inward radial force of at least 0.1 Newtons per cm.

15. The medical device of claim 11, wherein said composition-of-matter comprises a third composition being attached to at least a part of a surface thereof.

16. A method of lining a blood vessel, the method comprising introducing the medical device of claim 11 into the blood vessel.

17. The method of claim 16, further comprising imaging at least a part of said blood vessel during said introducing the medical device to the blood vessel.

18. A process of producing the composition-of-matter of claim 1, the process comprising:
mixing said at least one generally nondistensible polymer and said agent so as to provide a liquefied mixture; and
electrospinning said liquefied mixture onto a precipitation electrode such as to form said at least one plastically deformable fiber, thereby forming the composition-of-matter.

19. The process of claim 18, wherein said precipitation electrode comprises a rotating mandrel, thereby forming a tubular structure.

20. The process of claim 18, further comprising applying a thermal treatment to said tubular structure.

21. The process of claim 18, wherein said thermal treatment is selected so as to enhance radial strength of said tubular structure.

22. The process of claim 18, wherein said thermal treatment is selected so as to enhance anti-kinking resistance of said tubular structure.

23. The process of claim 18, wherein said thermal treatment comprises placing said tubular structure on a thermally isolated substrate and heating said tubular structure.

24. The process of claim 18, wherein said thermal treatment comprises rolling said tubular structure on a heated plate.

25. The process of claim 18, further comprising supplementing said liquefied polymer with a charge control agent, prior to said electrospinning.
